(19) United States
(12) Patent Application Publication
(10) Pub. No.: US 2018/0344590 A1
(43) Pub. Date: Dec. 6, 2018

(54) CONTROL OF EVAPORATION OF EMULSIONS STABILIZED WITH LIGNIN

(71) Applicants: Centre national de la recherche scientifique, Ange (FR); UNIVERSITE D'ORLEANS, Orleans (FR)

(72) Inventors: Arayik HAMBARDZUMYAN, Orleans (FR); Fabienne MEDUCIN-MICHAUD, Fleury Les Aubrais (FR); Samuel GUILLOT, Allonnes (FR)

(21) Appl. No.: 15/779,842

(22) PCT Filed: Nov. 28, 2016
(86) PCT No.: PCT/EP2016/078996

§ 371 Date: May 29, 2018

(30) Foreign Application Priority Data
Nov. 30, 2015 (FR) 1561574

(51) Int. Cl. A61K 8/06; B01F 17/00; A61K 8/84; A61K 8/19; A61Q 19/00

(52) U.S. Cl. CPC A61K 8/062; B01F 17/0028; B01F 17/0007; A61Q 19/00; A61K 8/84; A61K 8/19; B01F 17/0085

(57) ABSTRACT

The use of lignin in an oil-in-water emulsion, the oil phase representing at least 50 wt. % of the emulsion, in order to delay the evaporation of the oil phase and/or increase the mechanical strength of the adsorption layer formed by the lignin.

The present invention relates to emulsions, in particular oil-in-water type emulsions, and, more specifically, to a method for limiting the rate of evaporation of the oily phase in these emulsions.

### INTRODUCTION

Emulsions are widely produced and used in industry either as materials to be consumed or to be applied to surfaces as non-water soluble agents. We find emulsions in pharmaceuticals, in cosmetics (milks, creams, ointments), in cooking (sauces, creams), in painting, in the road industry, in agrochemicals, in detergents, in rolling mills, in the iron and steel industry, and in manufacturing various coatings (printing, adhesives . . . ).

In cosmetics and pharmacy, emulsions are an effective way to obtain a harmonious combination of ingredients with a different nature and properties, in particular lipophilic and hydrophilic ingredients, in a homogeneous and easy-to-use form.

An emulsion may be defined as an intimate mixture of two immiscible liquid substances, consisting of an aqueous phase and an oily or fatty phase. By mechanical and/or chemical action, the creation of an emulsion makes it possible to mix these two phases, wherein one of the phases is said to be “dispersed” in the second phase in the form of small droplets.

There are emulsions of the type “oil-in-water” and “water-in-oil”. Thus, an oil-in-water emulsion (O/W for oil-in-water) is composed of an oily phase dispersed in an aqueous phase. This is referred to as a “direct” emulsion.

Conversely, a water-in-oil emulsion (W/O for water-in-oil) is composed of an aqueous phase dispersed in an oily phase. A W/O emulsion is more fatty to the touch, because the feel mainly relates to the nature of the external phase. Such an emulsion is referred to as an “inverse” emulsion.

From a thermodynamic point of view, emulsions are inherently unstable, especially over time. The mixture remains stable thanks to the presence of a compound called an emulsifier that is capable of maintaining the stability of the mixture of both the aqueous and oily phases.

The emulsifiers may be ionic or nonionic in nature. The molecules used may be of natural or synthetic origin.

Emulsifiers are most often surfactants. The most widely used emulsifiers to date are alkyl sulfates and sulfonates, alcohols, acids, ethoxylated fatty esters, and sorbitan fatty esters.

The choice of a emulsifier to maintain the stability of a particular emulsion is based on many parameters.

Today, the use of molecules of natural origin is preferred.

In addition, given the deleterious effects of some emulsifiers on human or animal health, the current trend is to reduce the amounts of emulsifiers used.

Some emulsifiers offer specific advantages in addition to their stabilizing properties. Thus, certain emulsifiers protect the active ingredients against oxidation and/or against ultraviolet rays. Other emulsifiers make it possible to stabilize and preserve the physical and chemical integrity of the emulsions over very long periods of time.

Another advantageous property of emulsifiers is their ability to delay the evaporation of emulsions. Such emulsifiers having this additional advantage are actively sought.

It has been proposed to add wax to the emulsion in order to limit the evaporation of the aqueous phase of the emulsions (U.S. Pat. No. 5,780,409).

To limit the evaporation of the fatty phase in an oil-in-water emulsion, it has been proposed to use a polymer such as polyvinyl alcohol as the emulsifier, wherein the polymer makes it possible to thicken the layer of water on the surface and thus delay the evaporation of the fatty phase (Aranberri et al., 2003).

It has also been shown that the evaporation of the oily phase is delayed in the presence of an adsorption layer composed of silica nanoparticles. The evaporation rate of this phase may be further delayed by compressing the adsorption layer composed of these nanoparticles (Binks et al., 2010).

The solutions proposed above are not suitable for so-called “concentrated” emulsions, in which the weight of the oily phase is at least equal to the weight of the aqueous phase, or even greater than that of the aqueous phase.

The inventors have identified a means for delaying the evaporation of the fatty phase within concentrated oil-in-water emulsions by using a natural polymer, lignin.

The inventors have also identified a means for rendering the adsorption layers surrounding the oil droplets in concentrated oil-in-water emulsions, resistant to external mechanical stresses, in particular to a pressure exerted on the emulsion.

### SUMMARY OF THE INVENTION

The present invention relates to the use of lignin in a concentrated oil-in-water emulsion, characterized in that the oily phase represents at least 50% by weight of the emulsion, in order to delay the evaporation of the oily phase.

The present invention also relates to the use of lignin in a concentrated oil-in-water emulsion, characterized in that the oily phase represents at least 50% by weight of the emulsion, in order to increase the mechanical strength of the adsorption layer.

Of course, the two uses mentioned above may be complementary.

According to a particular aspect of the invention, the lignin is coupled to nanoparticles of gold and/or silver.

The present invention also relates to a method for preparing an oil-in-water type emulsion offering evaporation of the delayed oily phase, and comprising the following steps:
combination of an oily phase and an aqueous phase, wherein the oily phase represents at least 50% by weight of the emulsion;
addition of lignin coupled to nanoparticles of gold and/or silver, according to an oily/lignin phase ratio of between 50 and 10, preferably between 50 and 40;
emulsification by stirring.

The present invention also relates to a composition in the form of an oil-in-water type emulsion, wherein the oily phase represents at least 50% by weight of the emulsion, containing lignin in an oily/lignin phase mass ratio between 50 and 10, preferably between 50 and 40, wherein the lignin is coupled to gold and/or silver particles.

The present invention also relates to a composition in the form of an oil-in-water emulsion, wherein the oily phase represents at least 50% by weight of the emulsion, containing lignin in an oily/lignin phase mass ratio between 50 and 10, and wherein the lignin has been previously bleached by treatment with hydrogen peroxide.

Advantageously, these compositions, although concentrated, i.e. containing at least 50% by weight of oily phase, remain fluid thanks to the use of lignin as an emulsifier.

One of the major advantages of the invention is the use as emulsifier of a natural biodegradable molecule which has a low cost and a low impact on the environment, and which has additional properties of protection against evaporation and against mechanical shocks.

### DESCRIPTION OF THE FIGURES

FIG. 1: Evaporation curves, as a function of time, of the biphasic heptane/water system (6.5/3.5 v/v) and heptane/water emulsions stabilized with different surfactants:
1—biphasic n-heptane/water system without surfactant
2—n-heptane/water emulsion stabilized with 0.1 g of lignin
3—n-heptane/water emulsion stabilized with 0.1 g of triblock polymer surfactant PEO₁₀₆-PPO₃₀-PEO₁₀₆ (F-127)
4—n-heptane/water emulsion stabilized with 0.1 g of dodecyltrimethylammonium bromide (DTAB)

FIG. 2: Evaporation curves, as a function of time, of the biphasic decane/water system (6.5/3.5 v/v) and of the decane/water emulsions stabilized with different surfactants.
1—biphasic decane/water system without surfactant
2—decane/water emulsion stabilized with 0.1 g of lignin
3—decane/water emulsion stabilized with 0.1 g of triblock polymer surfactant PEO₁₀₆-PPO₃₀-PEO₁₀₆ (F-127)
4—decane/water emulsion stabilized with 0.1 g of dodecyl trimethyl ammonium bromide (DTAB)

FIG. 3: Evaporation curves, as a function of time, the biphasic dodecane/water system (6.5/3.5 v/v) and decane/water emulsions stabilized with different surfactants.
1—biphasic dodecane system without surfactant
2—dodecane/water emulsion stabilized with 0.1 g of lignin
3—dodecane/emulsion stabilized with 0.1 g of triblock polymer surfactant PEO₁₀₆-PPO₃₀-PEO₁₀₆ (F-127)
4—dodecane/water emulsion stabilized with 0.1 g of dodecyl trimethyl ammonium bromide (DTAB).

FIG. 4: Evaporation curves, as a function of time, of the previous biphasic systems and corresponding emulsions stabilized with 0.1 g of lignin
1—biphasic n-heptane/water system without surfactant
2—n-heptane/water emulsion stabilized with 0.1 g of lignin
3—decane/water emulsion stabilized with 0.1 g of lignin
4—biphasic decane/water system without surfactant
5—dodecane/water emulsion stabilized with 0.1 g of lignin
6—biphasic dodecane/water system without surfactant

FIG. 5: Optical microscopy photographs of the lignin adsorption layer in an emulsion composed of decane and water, in proportions of 65/35 volume/volume.
A—emulsion without physical constraint,
B—emulsion subjected to external physical constraint.

FIG. 6: Diagram of the Langmuir single layer technique.

FIG. 7: Schematic representation of a bubble tensiometer.
1—Optical bench, 2—Light source, 3—Thermostated and/or pressurized measuring cell, 4—Syringe pump, 5—Optics and camera, 6—Computer, 7—Control screen.

FIG. 8: Curve representing the shape of the apparent contour of the droplet, and the contact angles (θ, M) between the droplet and its support, wherein x and z are the Cartesian coordinates of each point on the profile of the bubble.

### DETAILED DESCRIPTION OF THE INVENTION

The present invention relates to a new use of lignin for delaying the evaporation of the oily phase of a concentrated emulsion of the oil-in-water type.

The present invention also relates to the use of lignin, for increasing the mechanical strength of the adsorption layer formed by the lignin around droplets of the dispersed oily phase of a concentrated emulsion of oil-in-water type.

The present invention is particularly useful for cosmetic applications. Many lipophilic compounds used in the formulation of cosmetic emulsions, such as perfumes, are fragile: they may have low stability, may be easily degraded and, in particular, have a tendency to evaporate rapidly.

In the emulsions according to the present invention, the release of the lipophilic active agents is controlled and delayed by the formation of an adsorption layer with a particular structure at the interface of the two aqueous and oily phases.

The terms used in this application are better defined below in order to promote good comprehension of the invention.

As indicated above, an emulsion is a dispersion, in the form of droplets, of two liquids that are immiscible with each other. It consists of an aqueous phase; and a so-called fatty, oily, organic or lipophilic phase, wherein these adjectives are used interchangeably and designate the same phase in the present application.

An oil-in-water emulsion comprises an oily phase dispersed in an aqueous phase.

An emulsion also comprises, in small amounts, an emulsifier also referred to herein as a “surfactant”, allowing the formation of adsorption layers at the interface of the two immiscible liquids.

For the purposes of the invention, the term “adsorption layer” designates the layer formed at the aqueous phase/oily phase interface in the emulsion, and making it possible to maintain the dispersion of the droplets of the oily phase in the aqueous phase. This layer is composed of lignin in this case.

Lignin

Lignin is one of the main components of wood, along with cellulose and hemicellulose. All vascular plants, woody and herbaceous, produce lignin.

Lignin is the second most abundant renewable biopolymer on Earth after cellulose. This polymer consists of at least three different types of monomers, i.e. the following monolignols:

coumaryl alcohol, called H unit (hydroxyphenyl), without methoxy group;

coniferyl alcohol, called G unit (guaiacyl), with one methoxy group;

sinapyl alcohol, called S unit (syringyl), with two methoxy groups.

The fraction of each monomer varies significantly as a function of the origin of the lignin: it differs according to the plant line (gymnosperm, monocotyledonous angiosperm, dicotyledonous angiosperm); of the species; of the organ; of the tissue; and, in a general way, of the physico-chemical environment in which the plant grows.

There is therefore no single and precise definition of lignin, because of its considerable variability even within a given species.

Lignin is known for its emulsion-stabilizing properties, as published by Rojas et al. (2007).

The lignin used for the implementation of the present invention is preferably a lignin of natural origin.

Lignins are preferably extracted from woody or herbaceous vascular plants by performing acidolysis in an organic solvent. For example, the extraction mode in a dioxane/water medium, in the presence of hydrochloric acid, makes it possible to obtain lignin preparations that are relatively unmodified and low in associated sugars (Monties, 1988).

According to a particular aspect of the invention, the lignin used is a by-product from plants of a cellulose extraction method.

According to another aspect of the invention, the lignin used for the implementation of the present invention is of synthetic origin.

Lignin of synthetic origin, also called ‘DHP’ for ‘dehydro-polymer’, may be synthesized in vitro in two stages:
(i) synthesis of coniferyl alcohol or other monolignol and
(ii) polymerization of monolignols in the presence of hydrogen peroxide and peroxidase.

According to a preferred embodiment of the invention, the lignin is bleached beforehand by treatment with hydrogen peroxide.

This eliminates the natural dark brown color of lignin which is not suitable for use in cosmetics.

Following treatment with hydrogen peroxide, consisting of a simple mixture of the two compounds in an aqueous solution, the lignin becomes white and may be incorporated into cosmetic compositions without changing the color of the composition.

According to a particular aspect of the invention, the oxygenated water is present at a concentration of 15% to 95% in the aqueous solution of lignin treatment.

According to another particular aspect of the invention, the treatment of lignin with hydrogen peroxide lasts one minute, two minutes, three minutes, four minutes, five minutes, ten minutes, fifteen minutes, twenty minutes, thirty minutes or one hour.

According to another particular aspect of the invention, the mixture of lignin and hydrogen peroxide is exposed to solar radiation for the duration indicated above, in order to accelerate the bleaching method.

Interestingly, it has been found that the surfactant properties of lignin are not affected by this treatment with hydrogen peroxide.

Delayed Evaporation

For the purposes of the invention, the term “delaying the evaporation of the oily phase” means decreasing the rate at which the oily phase evaporates over time.

The evaporation of this phase may be measured by various techniques that are well known to persons skilled in the art.

Advantageously, this technique is based on the use of a Langmuir-Blodgett tank that is used inside a closed chamber to eliminate any air flow and adjust the temperature to give a constant surface pressure and temperature.

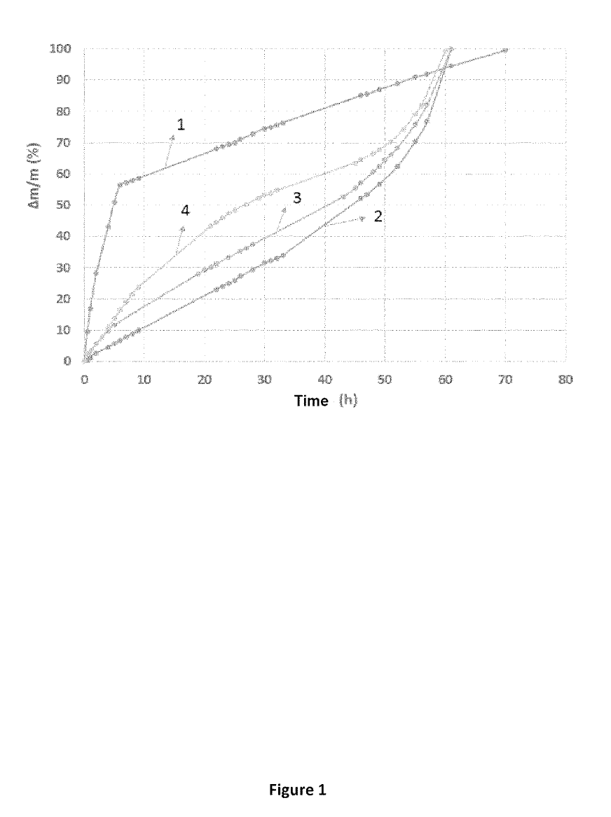

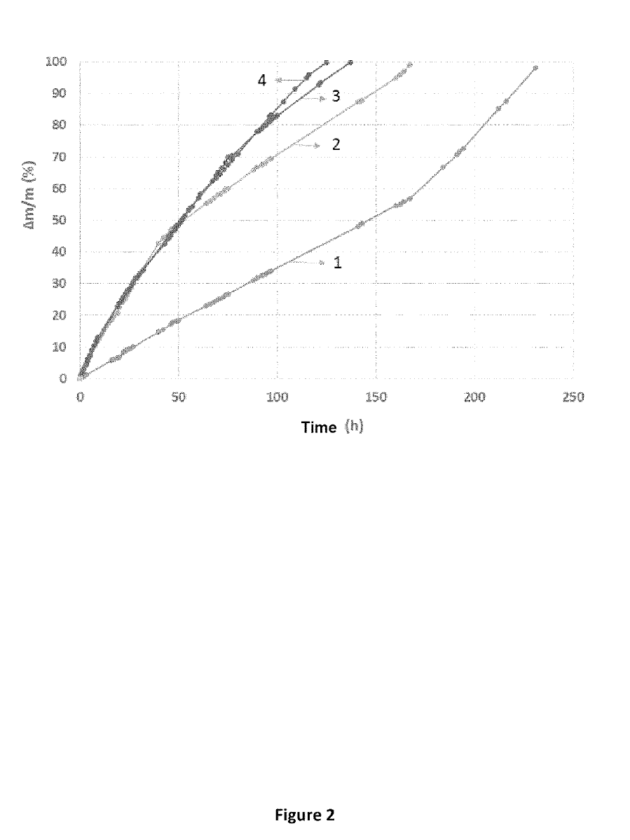

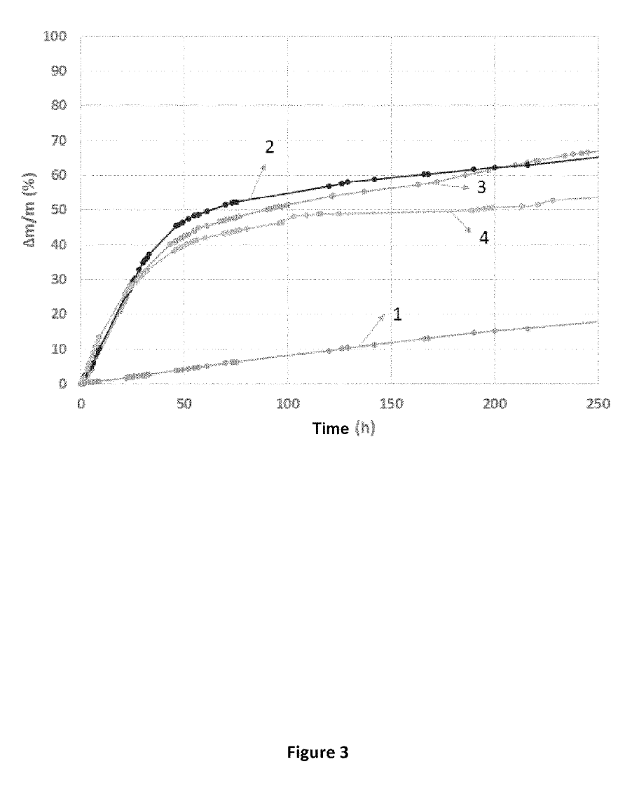

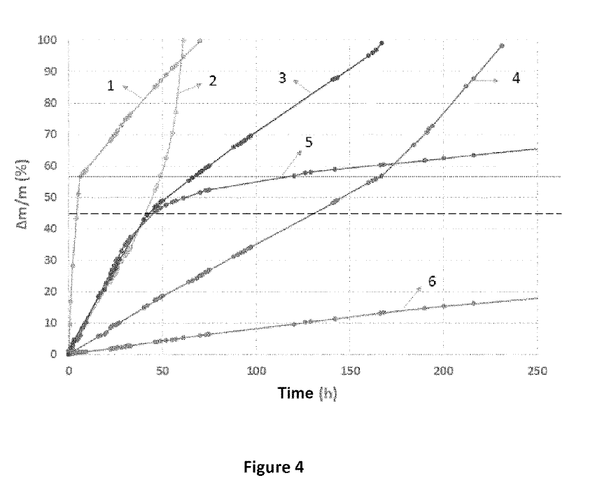

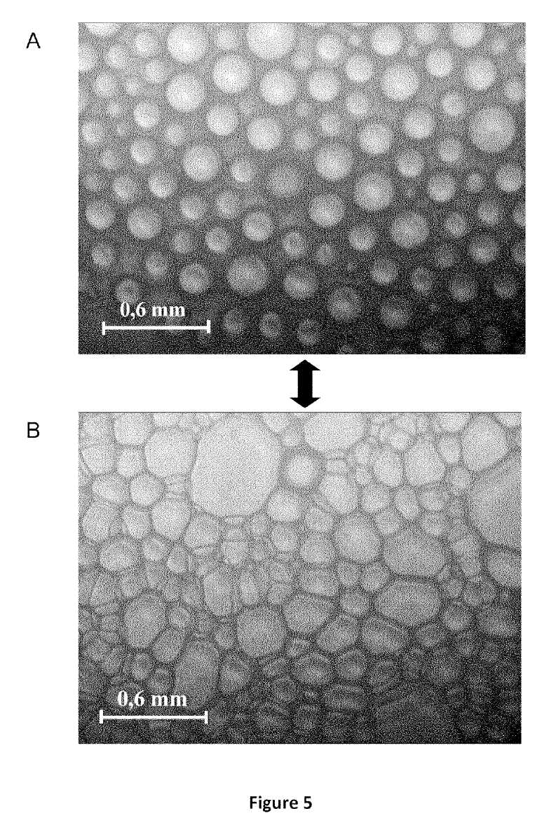

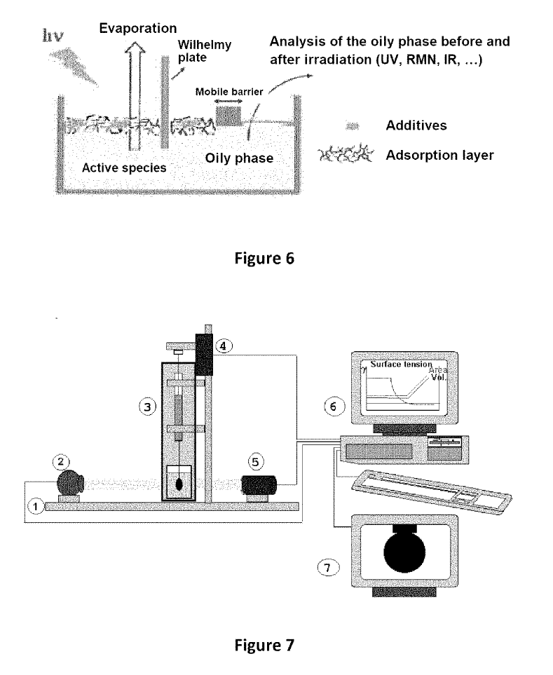

The Languir monolayer technique, as shown in FIG. 6, makes it possible to monitor the quantities of the volatile species by analytical methods of analysis before and after irradiation with UV (ultra-violet), IR (Infrared) and by NMR (nuclear magnetic resonance), in a Langmuir-Blodgett tank.

The kinetics of evaporation of the emulsion is measured over time. The evaporation rate is evaluated by weighing the flasks containing the emulsions at time T₀ then at time T₁, and the percentage of evaporation is calculated as a function of the weight difference measured between T₀ and T₁.

Increased Mechanical Strength

According to one aspect of the invention, the use of lignin in a concentrated emulsion makes it possible to increase the mechanical strength of the adsorption layer made of lignin.

Without wishing to be bound by the theory set out below, the inventors consider that the new particular properties observed when lignin is present in concentrated emulsions are related to the formation of adsorption layers having a specific structure. In fact, it appears that the lignin polymer forms a ‘three-dimensional network’, also called ‘grid’ or ‘matrix’, wherein the network allows interconnections between the adsorption layers present around each oily phase droplet. Thus, this ‘network’ offers surprising mechanical properties that prevent the settling or reduce the settling rate of the droplets of the dispersed phase, and reduce the rate of evaporation of the oily phase in the oil/water emulsion.

This mechanical property of the lignin adsorption layer is particularly advantageous for emulsions which are likely to be subjected to external pressures above atmospheric pressure. In particular, these emulsions may be stored in containers such as, for example, tubes, which will are subjected to pressure, in particular by the hand of the user, to allow the distribution of the emulsion.

Oily phase droplets, coated with a lignin adsorption layer, tend to be more pressure resistant than similar oily phase droplets coated with an adsorption layer of a distinct nature. This is demonstrated in the examples and, in particular, illustrated by the results presented in FIG. 5.

The mechanical properties of the adsorption layers are studied by techniques that are well known to persons skilled in the art, in particular by using devices for measuring the surface tension of liquids, such as the droplet tensiometer and the Langmuir balance.

A droplet tensiometer, also called a bubble tensiometer, is shown schematically in FIG. 7. With this device, a droplet of liquid is automatically formed at the end of the needle of a syringe in a bowl containing another liquid. The drop is illuminated by a uniform light source of the integrating sphere type, while the image of its profile is projected by an eclectic lens onto a CCD camera (512×512 pixels) and then digitized. The image obtained is then processed by software to determine, several times per second, the interfacial tension, the surface and the volume of the droplet.

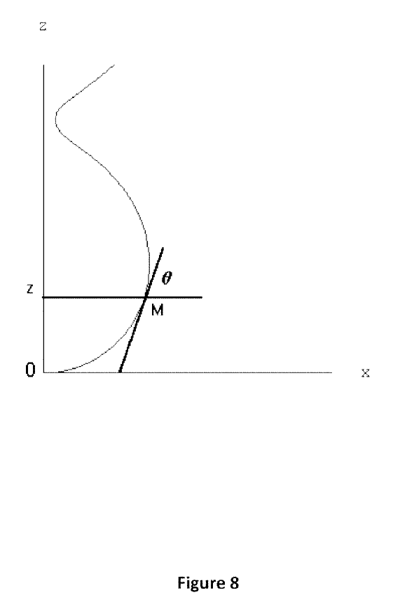

The computation of the interfacial tension is effected by the application of the Laplace Young equation, i.e. starting from the study of the contour of a drop having a symmetry of revolution. The shape of the droplet is determined by the combination of interfacial tension and the effects of gravitation. The effects of the interfacial tension force the droplet to adopt a spherical shape while the effects of gravitation tend to lengthen it and give it a pear shape in the case of a suspended droplet, and to flatten it into the case of a deposited droplet. When the importance of these effects is of the same order, the shape of the apparent contour and the contact angles between the droplet and its support may be determined (FIG. 8).

The data processing is based on two fundamental equations:
the Laplace-Young equation, which shows that the pressure difference caused by the curvature of the surface is proportional to the mean curvature, wherein the coefficient of proportionality is precisely the interfacial tension:

$$Δ P = γ ( \frac{1}{R} + \frac{1}{{R}^{′}} )$$

an equation that results from thermodynamic equilibrium.
the second equation results from the achievement of a balance of forces across any horizontal plane:

2πxγ sinθ=V(ρₖ−ρₗ)g+πx²p

where

p is the pressure due to the curvature

γ is the interfacial tension

R and R′ are the principal radii of curvature of the surface

x is the abscissa of the point of the ordinate meridian z

θ is the angle between the normal and the axis of revolution

V is the volume of the fluid under the plane

ph and pl are the respective densities of the two fluids, and

g is the terrestrial acceleration.

Another technique that is conventionally used to measure the surface pressure (ΔP) of the adsorption layers formed at the liquid/air and liquid/liquid interfaces is based on the use of the Langmuir balance.
The surface pressure is equal to ΔP=γ1-γ2,
   where γ1 is the surface tension of the pure solvent (without adsorption layer),
   and γ2 is the surface tension of the same solvent with the adsorption layer.
γ2 may be determined via the appreciation of the surface pressure of the liquid (ΔP) with the Langmuir balance, and by knowing the value of γ1.

Coupling Lignin with Nanoparticles

According to a particular aspect of the invention, the lignin used to stabilize the concentrated O/W emulsion, and to delay the evaporation of the oily phase and/or to increase the mechanical strength of the adsorption layer, is coupled with metallic nanoparticles.

These nanoparticles are, in particular, nanoparticles of gold and/or silver.

According to a first aspect of the invention, the lignin is coupled with gold particles.

According to a second aspect of the invention, the lignin is coupled with silver particles.

According to a third aspect of the invention, the lignin is coupled with a combination of gold particles and silver particles.

These nanoparticles are coupled with lignin, i.e. they are integrated in the lignin polymer constituting the adsorption layer, and are linked to the lignin molecules, either by covalent bonds or by noncovalent bonds.

In some embodiments, a portion of the metal nanoparticles is bonded to the lignin molecules of the adsorption layer by covalent bonds while the remaining portion of the metal nanoparticles is bonded to the lignin molecules of the adsorption layer through noncovalent bonds.

For the purposes of the invention, non-covalent bonds include weak bonds such as hydrogen bonds, ionic bonds and Van Der Waals interactions.

In order to couple metal nanoparticles to lignin molecules by covalent bonds, it is possible, in particular, to bind nanoparticles of gold and/or silver to the thiol functions present in the lignin molecule, according to methods known to persons skilled in the art (see for reference the article Jie Xu and Hu-Lin Li, 1995).

Advantageously, these nanoparticles are present in an amount of 10, 25 and 50 mg for a quantity of 100 mg of lignin. Thus, the nanoparticles are present in proportions relative to the total amount of lignin + nanoparticles, between 5% and 35% by weight, and more particularly in proportions of approximately 9%, 20% and 33% by weight.

Advantageously, the presence of these nanoparticles of gold and/or silver within the lignin polymer constituting an adsorption layer makes it possible to obtain adsorption layers having antioxidant properties.

Amount of Lignin in the Emulsion

One of the main advantages of using lignin as an emulsifier, as well as for the other properties mentioned above, is the possibility of using a reduced amount of lignin to durably stabilize the emulsions thus formed.

In particular, it has been observed that concentrated oil-in-water emulsions, comprising less than 5% by weight of lignin relative to the total weight of the emulsion, are stable for several months.

According to a particular aspect of the invention, the lignin is present in the emulsion in an amount of less than 5%, preferably less than 4%, 3% or 2% by weight, and more preferably in an amount of 0.5% to 2% by weight compared to the total weight of the emulsion.

It is understood that the terminal values of the indicated range are within the said range.

According to a particular aspect of the invention, the lignin is used in an amount of about 1% by weight relative to the total weight of the emulsion.

The use of lignin for the purposes claimed, namely the delaying of the evaporation of the oily phase, and/or the increase in the mechanical strength of the adsorption layer formed around the oily phase, is advantageously achieved under particular fatty/lignin phase mass ratio conditions for the oil-water emulsion in question.

Thus, it is preferable according to the invention, to use a quantity of lignin so that the ratio by weight of the oily phase with respect to the lignin is between 50 and 10, preferably between 50 and 30, more preferably between 50 and 40, even between 50 and 45.

Nature and Quantity of Oily and Aqueous Phases

The present invention relates to the use of lignin in a concentrated oil-in-water emulsion, wherein the oily phase represents at least 50% by weight of the emulsion, relative to the total weight of the emulsion.

The amount of oily phase in the emulsion according to the invention represents at least 50% by weight of the emulsion, i.e. it is either present at about 50%, or is present in a higher proportion than 50% by weight relative to the total weight of the emulsion.

In particular, the emulsion described in the invention comprises at least 50%, 55%, 60%, 65%, 70%, 75%, 80%, or 85% by weight of fatty phase, relative to the total weight of the emulsion.

The amount of oily phase in the emulsion according to the invention is, in particular, between 50 to 80% by weight and preferably 50 to 70% by weight, and more preferably between 50% by weight and 60% by weight, relative to the total weight of the emulsion.

According to a preferred aspect of the invention, the oily phase represents between 50 and 70% by weight of the emulsion.

According to the invention, the aqueous phase of the emulsion represents less than 50% by weight relative to the total weight of the so-called “concentrated” emulsion, preferably from 20% to 50% by weight, and better still from 40 to 50% by weight.

In some cases, the proportions of the different phases are expressed in volume relative to the total volume of the emulsion: it is then necessary to calculate, by virtue of the molecular weight of each of the phases, the weight (mass) of each of the phases in order to distinguish the proportion by weight of the phases and to determine whether they are as defined in the present invention.

For example, upon considering the emulsions as described in the article (Aranberri et al., 2003), wherein these are characterized by a volume of 10% of organic solvents such as heptane (C₇H₁₆), the latter having a weight/volume ratio of 0.6838 g/cm³, the relative mass of the oily phase in this emulsion is therefore 6.8% relative to the total weight of the emulsion.

The aqueous phase of the emulsion according to the invention mainly comprises water and, optionally, one or more compounds that are miscible with water. The aqueous phase may also comprise ionic species, pH regulators, and all the active, preservative and coloring ingredients that are water soluble or water dispersible.

The oily phase of the emulsion according to the invention is a fatty phase comprising at least one fatty substance chosen from fatty substances that are liquid at room temperature (20-25° C.) or oils, volatile or otherwise, of plant, mineral or synthetic origin and their mixtures. The term “oil” is understood to mean a fatty substance which is liquid at room temperature (25° C.). The oily phase may also include any usual liposoluble or lipodispersible additive.

For an application in the cosmetic field, these oils are chosen from physiologically acceptable oils.

As oils that may be used in the composition of the invention, mention may be made for example of:
hydrocarbon oils of animal origin;
hydrocarbon oils of vegetable origin;
esters and synthetic ethers, especially of fatty acids;
linear or branched hydrocarbons of mineral or synthetic origin, such as paraffin oils, volatile or not, and their derivatives;
partially hydrocarbon and/or silicone fluorinated oils;
silicone oils;
and their mixtures.

Among the hydrocarbons of formula CₙHₘ, where n and m are two whole numbers, linear, aromatic and cyclic hydrocarbons may be used, regardless of their boiling point.

Essential oils, in particular those extracted from plants, are preferred hydrocarbons for the implementation of the invention.

More particularly, hydrocarbons which may be present in the oily phase of the invention are cyclopentane (C₅H₁₀), hexane (C₆H₁₄), methylcyclohexane (C₇H₁₄), heptane (C₇H₁₆), decane (C₁₀H₂₂), dodecane (C₁₂H₂₆) and toluene (C₇H₈).

The oily phase in the emulsion according to the invention may be composed of a single oil, in particular a single hydrocarbon, or may also consist of a mixture of two, three or even four different oils.

Method for Preparing a Concentrated Emulsion

The present invention also relates to a method for preparing an oil-in-water type emulsion, comprising the following successive stages:
a) combining an oily phase and an aqueous phase, wherein the oily phase represents at least 50% by weight of the emulsion;
b) adding lignin coupled with gold and/or silver nanoparticles in an oil/lignin mass ratio of between 50 and 10;
c) emulsification by stirring.

The step a) of combining the oily phase and the aqueous phase should be in the most appropriate order, as is well known to persons skilled in the art, in particular by adding the aqueous phase to the oily phase.

The stirring emulsification step is preferably carried out with a magnetized bar, by ultrasound, or by any other stirring system, at a temperature ranging from 20° to 45° C.

Stirring is carried out for a minimum of 1 minute, and is preferably maintained for at least 3 minutes. The stirring may also be performed sequentially by successive jerks (up/off). The stirring time should be adjusted according to the total volume of the emulsion, in particular it should be lengthened if the volume of the emulsion is large.

Advantageously, the emulsion thus obtained exhibits the evaporation of the delayed oily phase, and the mechanical strength of the adsorption layer, which is increased compared to emulsions stabilized with other emulsifiers than lignin.

Composition in the Form of Concentrated Emulsion

The present invention also relates to a composition in the form of an oil-in-water emulsion, wherein the oily phase represents at least 50% by weight of the emulsion, and contains lignin in an oily phase/lignin mass ratio between 50 and 10, and wherein the lignin is coupled with gold and/or silver nanoparticles.

The present invention also relates to a composition in the form of an oil-in-water emulsion, wherein the oily phase represents at least 50% by weight of the emulsion, and contains lignin in an oily phase/lignin mass ratio between 50 and 10, and wherein the lignin is previously bleached by treatment with hydrogen peroxide.

Preferably, the said emulsion composition comprises, as oily phase, a single hydrocarbon selected from cyclopentane (C₅H₁₀), hexane (C₆H₁₄), methylcyclohexane (C₇H₁₄), heptane (C₇H₁₆), decane (C₁₀H₂₂), dodecane (C₁₂H₂₆) and toluene (C₇H₈).

According to a preferred aspect, the oily/lignin phase mass ratio of the composition is between 40 and 50, and is more preferably between 47 and 50.

According to another particular aspect of the invention, the lignin used in this composition is present in a relative mass quantity of approximately 1%, relative to the total weight of the emulsion.

According to another particular aspect of the invention, the lignin used in this composition is coupled with nanoparticles of gold and/or silver.

According to another particular aspect of the invention, the composition described above is intended to be used in cosmetics. It is then to be understood that all the constituents of the composition will be selected from physiologically acceptable constituents. In this case, the compositions according to the invention may be in all galenical forms of oil-in-water type emulsions, for example in the form of serum, milk or cream, and they will be prepared according to the usual methods.

Some of these compositions are, in particular, intended for topical application and may, in particular, constitute a dermatological or cosmetic composition, for example intended for care (anti-wrinkle, anti-aging, hydration, sun protection, etc.), the treatment, the cleaning and the makeup of keratin materials and, in particular, the skin, lips, hair, eyelashes, hair and nails of human beings.

### EXAMPLES

Materials and Methods

Products used

The products used in the examples below are all marketed by SIGMA-ALDRICH and are used without further purification. The lignin used is lignin of synthetic origin, marketed by SIGMA-ALDRICH under the reference “Lignin, alkali low sulfonate content 471003”

The pluronic polymer F-127 also named PEO₁₀₆-PPO₇₀-PEO₁₀₆ has the formula (H(OCH2CH2)₁₀₆(OCH2CHCH3)₇₀(OCH2CH2)₁₀₆0H).

The DTAB polymer (dodecyl trimethylammonium bromide) has the chemical formula C₁₂H₂₃-N(CH₃)₃-Br.

Preparation of the Heptane/Water Emulsion by Sonication and Monitoring of the Evaporation Rate

In 4 flasks weighed (m₀) and numbered from 1 to 4, are successively placed 6.5 ml of n-heptane and 3.5 ml of millipore water. 0.1 g of lignin, F-127 or DTAB are added to each of flasks 2, 3 and 4, respectively. Then, the contents of each flask are stirred by ultrasound (BIOBLOCK SCIENTIFIC brand Vibra cell 75115). The sonication conditions are as follows: for 7 minutes, 1 second up, 1 second off, with an amplitude of 30%, at T^{O}=25° C.

After stirring, the respective weights of the flasks are re-measured (m: mass of the flask with the organic phase, the aqueous phase and the surfactant) and the masses of the emulsions are calculated for each flask (mₑₘᵤₗ=m−m₀).

Then, the flasks are incubated at 20° C. and the changes (decreases) over time (mₜ) of the weight of each of these flasks are measured (Δm=mₑₘᵤₗ−mₜ). Then, the percentage of the evaporated liquid is calculated according to the following equation: (Δm/m)×100, over time.

The preparation and the monitoring of the evaporation rate of the decane/water and dodecane/water emulsions with the surfactants F-127 and DTAB are carried out identically, according to the procedure presented above.

Sample Preparation for AFM (Atomic Force Microscope)

A sample of 20 μl is gently deposited on the silicon wafer surface (0.5×′0.5 cm) before rotating it around its axis with a speed equal to 3000 rpm (spin-coating). During this rotation, the emulsion spreads to form a thin layer on the surface of the wafer. After evaporation of the organic and aqueous phases, a surfactant film is formed on the surface of the wafer, which may be studied by atomic force microscopy (AFM).

### Example 1

### Heptane/Water Emulsion

We studied the evaporation of heptane/water, decane/water and dodecane/water emulsions stabilized with various surfactants. In particular, in this study we used:
synthetic lignin;
the triblock polymer surfactant PEO₁₀₆-PPO₃₀-PEO₁₀₆ (technical name F-127);
and DTAB (dodecyl trimethylammonium bromide) C₁₂H₂₃—N(CH₃)₃—Br.

We used the same amount of each of these surfactants (0.1 g) to stabilize the emulsions.

In FIG. 1, the evaporation curves of the biphasic heptane/water system and the heptane/water emulsions stabilized with different surfactants are grouped together. FIG. 1 shows that emulsions stabilized with F-127 (3) or DTAB (4) evaporate significantly faster than the emulsion stabilized with lignin (2).

Moreover, this rate of evaporation is faster in the case of the emulsion stabilized with DTAB, for which the evaporation curve is similar to that of the biphasic heptane/water system without surfactant (1).

In other words, unlike the emulsion stabilized with lignin, in the case of emulsions stabilized with F-127 or DTAB, the evaporation of water and heptane are carried out at the same time and the phase evaporation can not be controlled.

Such a difference in evaporation rate of the stabilized emulsions with different types of surfactant could be related to the different structures of the adsorption layer formed with these surfactants at the heptane/water interface.

In this context, the structure of each of these layers after total evaporation of these emulsions was visualized by scanning electron microscopy (SEM).

The images of the adsorption layers formed by the surfactants at the heptane/water interface clearly indicate that:
the lignin forms an adsorption layer in the form of a “network” or “grid” which offers a mechanical strength greater than that of the adsorption layers usually known, and thus allows the protection of micro decane droplets against shocks.
on the contrary, the images of the adsorption layers formed by the surfactants F-127 and DTAB show smooth, non-entangled surfaces.
Moreover, in the case of emulsions stabilized with lignin, there is the presence of pores in the materials, which are much smaller than those for the other interfaces. In fact, in the case of emulsions stabilized with DTAB, the pores are non-existent, and the emulsions stabilized with F-127 have pores of intermediate size.

### Example 2

### Emulsion Decane/Water

In FIG. 2, the evaporation curves of the biphasic decane/water system (6.5/3.5 v/v) and the decane/water emulsions in identical proportions stabilized with different surfactants, are grouped together.

FIG. 2 shows that emulsions stabilized with F-127 (3) or DTAB (4) evaporate faster than the stabilized emulsion with lignin (2).

The biphasic decane/water system without surfactant (1) has a much lower evaporation rate than that observed for stabilized emulsions; this is explained by the fact that the organic phase of these emulsions evaporates first, according to the kinetics expected for the said non-emulsified organic phase alone.

The images of the adsorption layers obtained with the lignin, F127 and DTAB surfactants formed at the decane/water interface show that the layer obtained with the lignin has numerous asperities, whereas the layers formed with the F-127 or the DTAB are much smoother, and have no network or three-dimensional structure.

Moreover, in the case of emulsions stabilized with lignin, there is the presence of pores in the materials, which is much smaller than for the other interfaces. In fact, in the case of emulsions stabilized with DTAB, the pores are non-existent, while the emulsions stabilized with F-127 have pores of intermediate size.

### Example 3

### Dodecane/Water Emulsion

In FIG. 3, the evaporation curves of the biphasic dodecane/water system and of the dodecane/water emulsions stabilized with different surfactants are grouped together.

FIG. 3 shows that emulsions stabilized with F-127 (3) or DTAB (4) evaporate faster than the emulsion stabilized with lignin (2).

The biphasic dodecane/water system without surfactant (1) has a much lower evaporation rate than that observed for stabilized emulsions; this is explained by the fact that the organic phase of these emulsions evaporates first, according to the kinetics expected for the non-emulsified organic phase alone.

The images of the adsorption layers obtained with the lignin, F127 and DTAB surfactants formed at the dodecane/water interface show that the layer obtained with the lignin has numerous asperities, whereas the layers formed with the F-127 or the DTAB are much smoother, and have no network or three-dimensional structure.

Moreover, in the case of emulsions stabilized with lignin, there is the presence of pores in the materials, which is much smaller than for the other interfaces. In fact, in the case of emulsions stabilized with DTAB, the pores are non-existent, and the emulsions stabilized with F-127 have pores of intermediate size.

In conclusion, the comparison of the scanning electron microscopic images (SEM) of the adsorption layers after a total evaporation of the stabilized emulsions with different surfactants, shows a good correlation between the structure of these adsorption layers and the evaporation mechanisms of the organic and aqueous phases.

Table 1 below shows the percentages of organic phase, aqueous phase and lignin used in the emulsions presented above.

TABLE 1

| | | | Ratio of | Ratio of | |
|---|---|---|---|---|---|
| | | Ratio | organic | aqueous | Ratio of |
| | | lignin to | phase to | phase to | organic |
| Nature of | | total | total | total | phase to |
| the | Boiling point | weight of | weight of | weight of | lignin |
| organic | of the | the | the | the | by |
| phase | hydrocarbons | emulsion | emulsion | emulsion | weight |
| C₇H₁₆- | 98° C. | 1.24% | 55.25% | 43.50% | 44.55 |
| heptane | | | | | |
| C₁₀H₂₂- | 174° C. | 1.20% | 56.72% | 42.07% | 47.26 |
| decane | | | | | |
| C₁₂H₂₆- | 216° C. | 1.18% | 57.37% | 41.44% | 48.61% |
| dodecane | | | | | |

On the other hand, the porosity and the three-dimensional structure of the adsorption layers formed with lignin vary with the boiling points of the organic phases used in the emulsions, as presented in Table 1 above. So, this approach could be interesting in the field of preparation of porous materials, because it would allow control of the pore size.

### Example 4

### Comparison of Evaporative Kinetics of Emulsions and Unstabilized Biphasic Systems

The different curves obtained previously for the biphasic systems and the emulsions stabilized with lignin are grouped on the same diagram as presented in FIG. 4. This figure makes it possible to highlight two distinct evaporation kinetics, depending on the presence or absence of lignin.

In the absence of lignin (curves 1, 4 and 6, non-emulsified system), two successive kinetics of evaporation may be very clearly observed, wherein the change of the slope of the curve takes place at the moment when the evaporated mass of the system reaches a point between 55 and 57% (see dotted line). It should be noted that for the curve (6), this point is outside the graph. This point of “break in curve” corresponds to the moment when the entire organic phase, present in a mass of 55 to 57%, is evaporated. It is understandable that this organic phase evaporates first, as this phase is the lightest and is therefore located above the aqueous phase.

In the presence of lignin (curves 2, 3 and 5), the evaporation mechanism is completely changed. Evaporation begins at a constant speed until the evaporated mass of the emulsion equals 43-45% of the total mass of the system (dashed line). Then, for each emulsion, the evaporation continues at a speed substantially equal to that of the organic phases present; this becomes clear by comparing the curves in pairs:
the slope of the beginning (before the point of change) of the curve 1 is the same as the slope of the curve 2, after the point of change;
the slope of the beginning of curve 4 is the same as the slope of curve 3 after the point of change;
the slope of curve 6 is the same as the slope of curve 5, after the point of change.

These observations demonstrate that the evaporation of the emulsions stabilized with lignin is first effected by the evaporation of the aqueous phase, and that the organic phase begins to evaporate only after complete evaporation of the aqueous phase.

This surprising result is probably related to the three-dimensional structure of the lignin-containing adsorption layers, which interconnect and form a three-dimensional network within the emulsion.

### Example 5

### Resistance of the Adsorption Layer to External Physical Stresses

FIG. 5 shows photos of the lignin-containing adsorption layer, under optical microscopy, before (A) and during (B) the application of a pressure important mechanics on the emulsion.

It is clear from these photos that the droplets resist this pressure, and come back into place as soon as the pressure subsides.

### BIBLIOGRAPHIC REFERENCES

U.S. Pat. No. 5,780,409
Ibon Aranberri, Bernard P. Binks, John. H. Clinta and Paul D. Fletcher. Delay of oil drop evaporation from oil-in-water emulsions. Chem. Commun., 2003, 2538-2539
Bernard P. Binks, Paul D. 1. Fletcher and Benjamin L. Holt. Selective Retardation of Perfume Oil Evaporation from Oil-In Water Emulsions Stabilized by Either Surfactant Gold Nanoparticles. Langmuir, 2010, 26 (23), pp 18024-18030
Rojas O.; Bullon J; Ysambertt F; Forgiarini A; Salter J L.; Argyropoulos D. Lignins as emulsion stabilizers; ACS symposium series 2007, vol. 954, pp. 182-199
Monices, 8. Preparation of dioxane lignin fractions by acidolysis. 1988 In Methods in enzymology.
JIE XU and HU-LIN LI, The Chemistry of Self-Assembled Long-Chain Alkanethiol Monolayers on Gold. 1995, Journal of Colloid and Interface Science, 176, 138-149.

1. A method to delay the evaporation of the oily phase of an oil-in-water emulsion comprising adding of lignin into the oil-in-water emulsion, wherein the oily phase represents at least 50% by weight of the emulsion, thereby delaying the evaporation of the oily phase.

2. A method to increase the mechanical strength of the adsorption layer of an oil-in-water emulsion comprising adding lignin in the oil-in-water emulsion, wherein the oily phase represents at least 50% by weight of the emulsion, thereby increasing the mechanical strength of the adsorption layer.

3. The method according to claim 1, wherein the lignin is of natural origin.

4. The method according to claim 3, wherein the lignin is a byproduct of a method for extracting cellulose from plants.

5. The method according to claim 1, wherein the lignin is of synthetic origin.

6. The method according to claim 1, wherein the lignin is previously bleached by treatment with hydrogen peroxide.

7. The method according to claim 1, wherein the lignin is coupled with nanoparticles of gold and/or silver.

8. The method according to claim 1, wherein the lignin is present in the emulsion in an amount of less than 5% by weight, compared to the total weight of the emulsion.

9. The method according to claim 1, wherein the ratio by weight of the oily phase on the lignin is between 50 and 10.

10. The method according to claim 1, wherein the oily phase represents between 50 and 70% by weight of the emulsion.

11. The method according to claim 1, wherein the oily phase comprises at least one hydrocarbon selected from the group consisting of cyclopentane, hexane, methylcyclohexane, heptane, decane, dodecane and toluene.

12. A method for preparing an oil-in-water type emulsion comprising the following successive steps:
a) combining an oily phase and an aqueous phase, wherein the oily phase represents at least 50% by weight of the emulsion;
b) adding lignin coupled with nanoparticles of gold and/or silver, according to an oily/lignin phase mass ratio between 50 and 10; and
c) emulsifying by stirring.

13. A composition in the form of an oil-in-water type emulsion, wherein the oily phase represents at least 50% by weight of the emulsion, containing lignin in an oily/lignin phase mass ratio of between 50 and 10, and wherein the lignin is coupled with gold and/or silver nanoparticles.

14. A composition in the form of an oil-in-water emulsion, wherein the oily phase represents at least 50% by weight of the emulsion, containing lignin in an oily phase/lignin mass ratio of between 50 and 10, and in which the lignin was previously bleached by treatment with hydrogen peroxide.

15. The method according to claim 2, wherein the lignin is of natural origin.

16. The method according to claim 15, wherein the lignin is a byproduct of a method for extracting cellulose from plants.

17. The method according to claim 2, wherein the lignin is of synthetic origin.

18. The method according to claim 2, wherein the lignin is previously bleached by treatment with hydrogen peroxide.

19. The method according to claim 2, wherein the lignin is coupled with nanoparticles of gold and/or silver.

20. The method according to claim 2, wherein the lignin is present in the emulsion in an amount of less than 5% by weight, compared to the total weight of the emulsion.

21. The method according to claim 2, wherein the ratio by weight of the oily phase on the lignin is between 50 and 10.

22. The method according to claim 2, wherein the oily phase represents between 50 and 70% by weight of the emulsion.

23. The method according to claim 2, wherein the oily phase comprises at least one hydrocarbon selected from the group consisting of cyclopentane, hexane, methylcyclohexane, heptane, decane, dodecane and toluene.
